(19) United States
(12) Patent Application Publication
(10) Pub. No.: US 2012/0035723 A1
(43) Pub. Date: Feb. 9, 2012

(54) PROSTHESIS FOR MITRAL ANNULOPLASTY

(76) Inventors: Philippe Primo Caimmi, Novara (IT); Giovanni Vacca, Novara (IT)

(21) Appl. No.: 13/263,971

(22) PCT Filed: Apr. 2, 2010
(86) PCT No.: PCT/EP10/02135

§ 371 Date: Oct. 11, 2011

(30) Foreign Application Priority Data
Apr. 8, 2009 (IT) TO2009A000270

(51) Int. Cl. A61F 2/24

(52) U.S. Cl. 623/2.37

(57) ABSTRACT

A prosthesis (10) for mitral annuloplasty, comprising an annular structure (11), in which the annular structure is composed of a flexible support segment (12), adapted to be fixed by suture along a posterior portion (PMA) of the mitral annulus (MA) which extends from the anterolateral trigone (AT) to the posteromedial trigone (PT), and of an intertrigonal rigid segment (13) interconnecting the ends (12a, 12b) of the flexible support segment. The intertrigonal rigid segment is adapted to act as a spacer between the anterolateral trigone and the posteromedial trigone. The annular structure is adapted to be fixed to the mitral annulus exclusively through the flexible support segment.

The present invention refers to a prosthesis for mitral annuloplasty, of the type comprising an annular structure.

Annuloplasty is a surgical technique allowing mitral valve insufficiency to be corrected.

Mitral valve insufficiency is caused by an incomplete coaptation of the mitral valve leaflets: the anterior leaflet and the posterior one. This incomplete coaptation can be due to alterations of the leaflets (fibrosis, prolapse, laceration), to dilation of the valve annulus that takes away the two leaflets, or to both phenomena. In all these circumstances, surgical correction consists of the surgical repair of the mitral annulus, or annuloplasty, or of the reduction of the perimeter of the mitral valve. This is very useful in increasing coaptation of the two leaflets, either when structural leaks due to the leaflets impose narrowing of the annulus in order to facilitate coaptation of the residual and properly manipulated leaflet portions, or when dilation of the mitral annulus turns out to be the main cause of mitral insufficiency. In order to make and stabilize such a narrowing of the mitral annulus, that is the annuloplasty, advantage is taken of artificial support members that are fixed by suture to the mitral annulus once it has been reduced.

The prostetic rings for the mitral valve are prostheses very used in repair surgery of the mitral valve. The use of prostetic rings and the repair surgery of the mitral valve represent without doubt the standard of reference of the surgical treatment of mitral pathology. Mitral repair surgery is a very used process both in the developed countries and in the developing ones, both in young and aged patients.

Like all prostheses, also mitral rings have the problem of preserving at its best physiology of the structure to be replaced or supported. With the aim of solving this functional problem, several types of mitral rings have been developed and introduced in the clinical use. Initially, rigid rings (such as Carpentier-Edwards Classic® rings) have been developed, which allow a good stabilization of the mitral repair to be obtained, but they eliminate the function of the mitral annulus and hardly reduce that of the aortic valve annulus.

FIG. 1 shows a view of the mitral valve MV from the left atrium. AL and PL indicate the anterior leaflet and the posterior leaflet, respectively, of the mitral valve, and MA indicates the mitral annulus. CT indicates the heart strings, while the zones marked by the dashed circles APM and PPM represent the positions of the anterolateral papillary muscle and of the posteromedial papillary muscle, respectively. AT and PT indicates the anterolateral trigone and the posteromedial trigone, respectively, while IT indicates the intervalvular trigone. Beyond the intervalvolar trigone, there is the aortic valve, not shown in the figure. Arrows SD in FIG. 1 represent the movement of the anterior portion of the mitral annulus (and, correspondingly, of the adjacent part of the aortic annulus) during the cardiac cycle. Such a movement is hindered by conventional rigid rings, with the resulting alteration of the dynamics of the mitral annulus and the adjacent aortic annulus.

Subsequently to rigid rings, flexible (such as Cosgrove-Edwards® ring) or semi-rigid rings (such as Carpentier-Edwards Physio® ring) have been conceived, with the aim of reducing negative impact on physiology of the aortic annulus. However, these rings do not guarantee the containment effectiveness of a rigid ring and have, like all rings, a negative impact on physiology of the aortic ring.

Rigid or flexible C-shaped prostheses have been therefore developed, with the aim of avoiding limitation of the dynamics of the adjacent aortic ring. For example, a prosthesis of this type is described in WO 02/074197. Such prostheses are very discussed, since recent studies (Circulation. 2005; 112 [suppl. I]: I-409-I-414) have shown that also the intertrigonal part of the anterior portion of the mitral annulus undergoes dilation processes.

WO-97/19655 describes a prosthesis for mitral annuloplasty having an annular structure, comprising a flexible support segment adapted to be fixed by suture along a posterior portion of the mitral annulus, and a rigid segment interconnecting the ends of the flexible support segment. The rigid segment is intended to be removed when the flexible segment has been fixed to the mitral valve, and serves only to keep the flexible segment in a proper configuration when it is moved and implanted. Therefore, it has only the function of a temporary spacer of the ends of the flexible segment.

An object of the present invention is that to propose a prosthesis for mitral annuloplasty that is able to overcome the above mentioned drawbacks, and that is able, in particular, to limit degenerative processes of dilation of the mitral annulus, without altering dynamics of the anterior portion of such a ring and of the adjacent aortic ring.

Such an object is reached according to the invention by a prosthesis for annuloplasty of the type defined at the beginning, in which said annular structure is composed of a flexible support segment, which is adapted to be sutured along a posterior portion of the mitral annulus extending from the anterolateral trigone to the posteromedial trigone, and of a non-removable rigid intertrigonal segment inseparably interconnecting the ends of the flexible support segment, said rigid intertrigonal segment being adapted to act as a spacer between said anterolateral trigone and posteromedial trigone, and in which said annular structure is adapted to be fixed to the mitral annulus exclusively through the flexible support segment.

In a prosthesis made according to such an idea of solution, by virtue of the combination of the flexible support segment with the intertrigonale rigid segment, degenerative dilation processes can be limited without altering physiology of the mitral annulus. In particular, no bond is imposed to dynamics of the anterior portion of the mitral annulus, which allows a normal mobility of the anterior leaflet and the adjacent aortic valvular annulus to be achieved. Moreover, it is possible to obtain a higher speed of implantation, since only suture of the posterior portion of the mitral annulus is required.

Particular embodiments of the invention are the subject of the dependent claims, the content of which is considered to be an integral part of the present description.

Further characteristics and advantages of the invention will appear from the following detailed description, provided purely as a non-limitative example, with reference to the appended drawings, in which:

FIG. 1 is a view of the mitral valve from the left atrium;

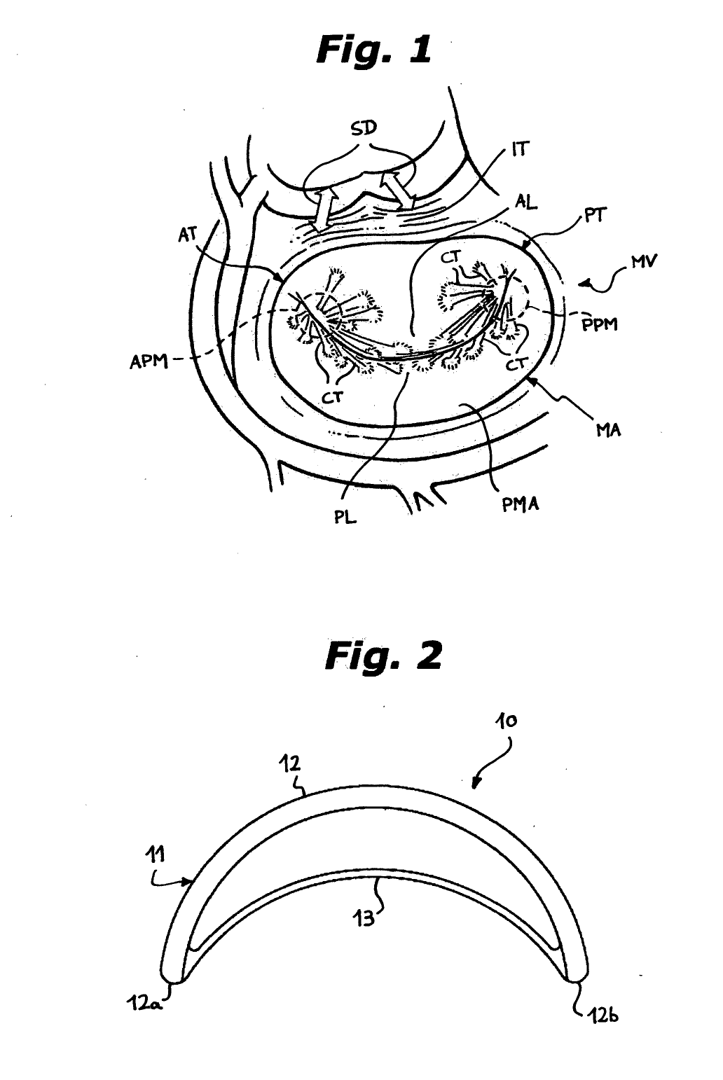

FIG. 2 is a simplified elevational view of a prosthesis according to the invention;

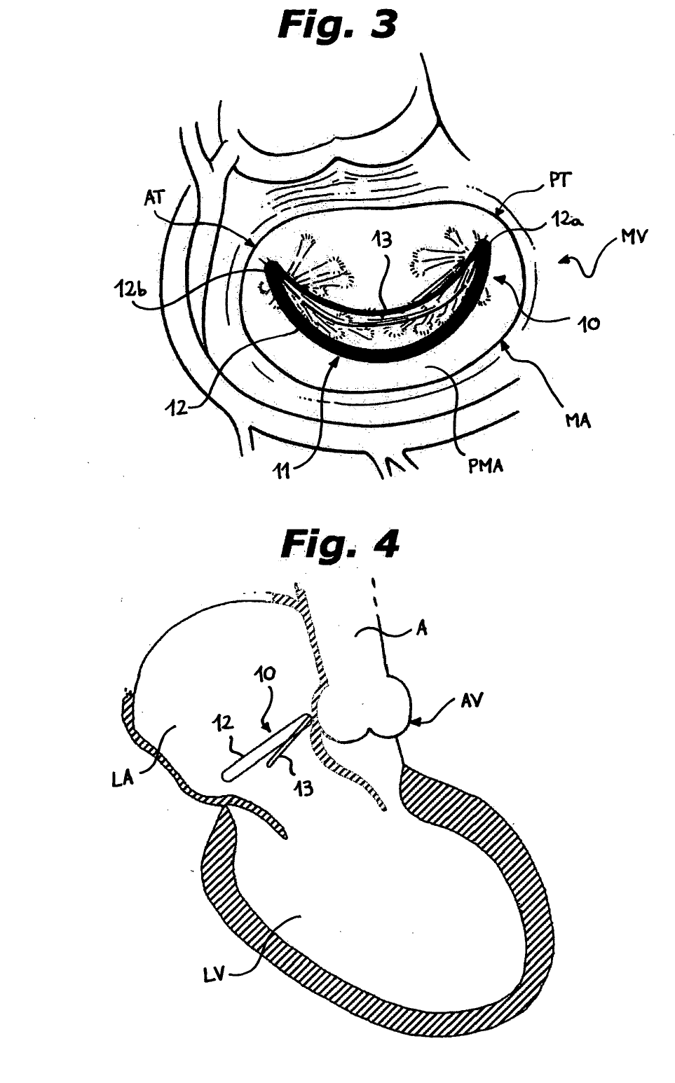

FIG. 3 is a view analogous to that of FIG. 1, in which the prosthesis of FIG. 2 in its implanted condition is also shown;

FIG. 4 is a sagittal section of the left ventricle, with the prosthesis of FIG. 2 implanted in its annular position; and

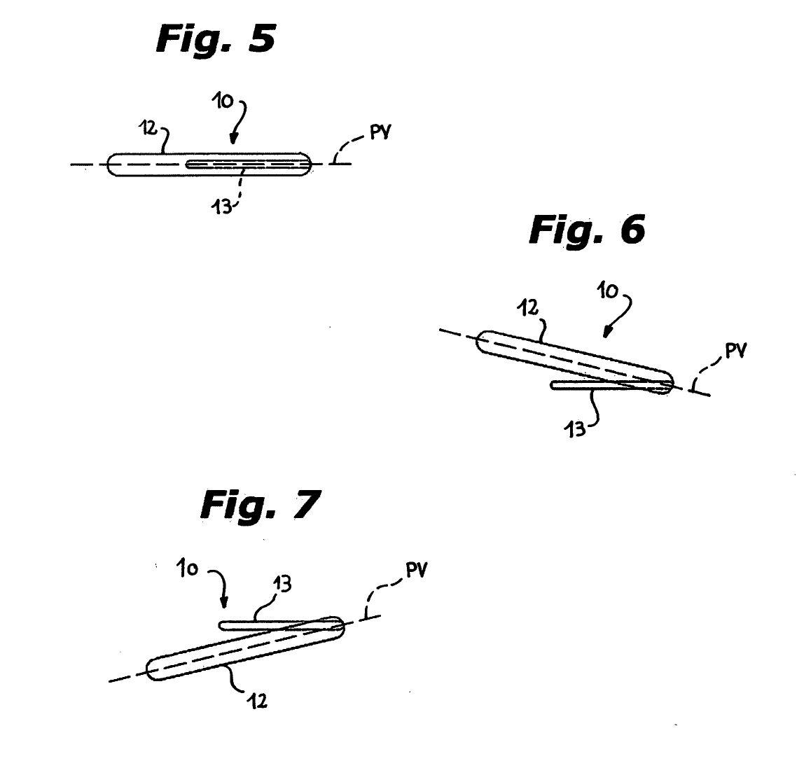

FIGS. 5 to 7 are side elevational simplified views of the prosthesis of FIG. 2, in different positions associated with the movement of the valvular plane.

With reference to FIGS. 2 and 3, a prosthesis for mitral annuloplasty is indicated 10 in its whole. Such a prosthesis comprises an annular structure 11, which is composed of a flexible support segment 12 and of a rigid intertrigonal segment 13, connected to each other so as to form a closed ring.

The flexible support segment 12 is intended to be fixed by suture along a posterior portion PMA of the mitral annulus which extends from the anterolateral trigone AT to the posteromedial trigone PT. The type of suture is not essential for the invention, provided that it is located along the whole extension of the flexible support segment 12.

The support segment 12 is flexible to such an extent to comply, in the use, with movements of the posterior portion of the mitral annulus. Moreover, it is substantially inextensible with respect to stresses transmitted in the use by the posterior portion PMA of the mitral annulus, in such a manner that it performs an effective limitative action.

For example, the flexible support segment 12 may consist of a body (for example a tube) of textile material fixed at its ends to the rigid intertrigonal segment 13, or by a structure comprising a core (for example of metallic wire) connected at its ends to the rigid intertrigonal segment 13 and enclosed by a sheathing of textile material.

The intertrigonal rigid segment 13 interconnects the ends 12a, 12b of the flexible support segment 12, and is adapted to act as a spacer between the anterolateral trigone AT and the posteromedial trigone PT of the mitral valve. Therefore, rigidity of the intertrigonal segment 13 must be sufficient to withstand the stresses deriving from surrounding cardiac tissue, in such a manner that the intertrigonal distance does not change in a significant manner also in the case of dilation processes of the anterior portion of the mitral annulus.

For example, the rigid intertrigonal segment 13 is of metallic material.

Preferably, the rigid intertrigonal segment 13 is bowed and is arranged in such a manner to project, in the use, towards the posterior portion PMA of the mitralic ring, as shown in FIG. 3. In other words, the intertrigonal segment 13 and the support segment 12 form, in the use, two arches the concavities of which are directed concordantly, in an elevational view. This configuration allows the blood flow passing through the mitral valve to be distributed in the best manner.

In the use, the intertrigonal segment 13 is intended to be free to rotate about its pivot points consisting of the ends 12a and 12b of the support segment 12, since the annular structure 11 of which it is part, is adapted to be fixed to the mitral annulus MA exclusively through the flexible support segment 12; in fact, the suture joining the device 10 to the mitral annulus is only applied on the flexible support segment 12, which therefore remains bound to the posterior portion of the mitral annulus.

A sagittal section of the left ventricle LV, with the prosthesis 10 implanted in its annular position, is shown in FIG. 4 in a simplified manner. The left atrium and the aorta are indicated LA and A, respectively, while the aortic valve is indicated AV.

The operation of the prosthesis according to the invention is similar to the static configuration of the double-hinged arch type. FIGS. 5 to 7 are side elevational views showing several positions assumed by the prosthesis 10 as a result of the change of attitude of the valvular plane (represented by the dashed line PV). As it can be noticed, while the flexible support member 12 follows the movement of the valvular plane PV, the rigid intertrigonal member 13 is free to oscillate in order to change its own tilting with respect to the valvular plane PV.

As it will be noticed, by virtue of the combination of the flexible support segment (fixed to the posterior portion of the mitral annulus) with the intertrigonal rigid segment (free to rotate with respect to the pivot points 12a and 12b) the degenerative dilation process can be limited without altering physiology of the mitral annulus. In particular, no bond is imposed to dynamics of the anterior portion of the mitral annulus, which allows a normal mobility of the anterior leaflet and the adjacent aortic valvular annulus to be achieved. Moreover, it is possible to obtain a higher speed of implantation, owing to the fact that only suture of the posterior portion of the mitral annulus is required.

1. A prosthesis for mitral annuloplasty comprising an annular structure, wherein said annular structure is composed of a flexible support segment, which is adapted to be sutured along a posterior portion (PMA) of the mitral annulus (MA) extending from the anterolateral trigone (AT) to the posteromedial trigone (PT), and of a non-removable rigid intertrigonal segment inseparably interconnecting the ends of the flexible support segment, said rigid intertrigonal segment being adapted to act as a spacer between said anterolateral trigone and posteromedial trigone, in which said annular structure is adapted to be fixed to the mitral annulus exclusively through the flexible support segment.

2. A prosthesis according to claim 1, wherein said flexible support segment is flexible to such an extent to comply in use with movements of the posterior portion of the mitral annulus.

3. A prosthesis according to claim 2, wherein said flexible support segment is substantially inextensible with respect to stresses transmitted thereto, in use, by the posterior portion of the mitral annulus.

4. A prosthesis according to claim 1, wherein said rigid intertrigonal segment is bowed and is arranged in such a manner as to project, in use, toward the posterior portion of the mitral annulus.

5. A prosthesis according to claim 1, wherein said flexible support segment is fixed at its ends to the rigid intertrigonal segment.

6. A prosthesis according to claim 5, wherein said flexible segment is made of textile material.

7. A prosthesis according to claim 1, wherein said flexible segment includes a core connected at its ends to the rigid intertrigonal segment, said core being contained in a sheathing of textile material.

8. A prosthesis according to claim 7, wherein said core is made of metal wire.

9. A prosthesis according to claim 1, wherein said rigid intertrigonal segment is made of metallic material.

10. A prosthesis according to claim 2, wherein said flexible segment includes a core connected at its ends to the rigid intertrigonal segment, said core being contained in a sheathing of textile material.

11. A prosthesis according to claim 10, wherein said core is made of metal wire.

12. A prosthesis according to claim 2, wherein said rigid intertrigonal segment is made of metallic material.

13. A prosthesis according to claim 3, wherein said flexible segment includes a core connected at its ends to the rigid intertrigonal segment, said core being contained in a sheathing of textile material.

14. A prosthesis according to claim 13, wherein said core is made of metal wire.

15. A prosthesis according to claim 2, wherein said rigid intertrigonal segment is made of metallic material.

16. A prosthesis according to claim 4, wherein said flexible segment includes a core connected at its ends to the rigid intertrigonal segment, said core being contained in a sheathing of textile material.

17. A prosthesis according to claim 16, wherein said core is made of metal wire.

18. A prosthesis according to claim 2, wherein said rigid intertrigonal segment is made of metallic material.
